(19) United States
(12) Patent Application Publication
(10) Pub. No.: US 2007/0224260 A1
(43) Pub. Date: Sep. 27, 2007

(54) Dosage Form Having Polymorphic Stability

(75) Inventors: Pavak R. Mehta, Noida (IN); Indu Bhushan, Hyderabad (IN); Pasula Basavaiah Chowdary, Krishna (IN); Kiran Krishnan, Palatine, IL (US); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: DR. REDDY'S LABORATORIES LIMITED, Hyderabad 500 016, A. P. (IN); DR. REDDY'S LABORATORIES, INC., Bridgewater, NJ (US)

(21) Appl. No.: 10/599,907

(22) PCT Filed: Apr. 14, 2005
(86) PCT No.: PCT/US05/12544

§ 371 Date: Oct. 13, 2006

Related U.S. Application Data
(60) Provisional application No. 60/600,332, filed on Aug. 9, 2004.

(30) Foreign Application Priority Data
Apr. 15, 2004 (IN) 346/CHE/2004

(51) Int. Cl. A61K 9/48; A61K 9/20

(52) U.S. Cl. 424/452; 424/451; 424/464

(57) ABSTRACT

Mini-tablets comprising a drug substance are formed by compression, using forces sufficiently low to preserve the polymorphic form of the drug. The mini-tablets can be administered directly, filled into capsules, etc.

### INTRODUCTION TO THE INVENTION

The present invention relates to a solid pharmaceutical dosage form for oral administration, containing a drug substance that is subject to polymorphic conversion during formulation procedures, or conversion during storage after formulation.

In some instances, different crystalline forms of drug substances have useful properties that are not found in other crystalline forms. These can include a desirable dissolution profile, enhanced particle flowability, and/or other properties. A difficulty in using polymorphic forms that are not the thermodynamically most stable form of a compound arises when the desired material spontaneously converts to the less desirable, more stable, form or when the polymorphic change occurs during formulation processing steps. In some cases, the spontaneous conversion is relatively slow, and occurs upon exposure of the substance to moisture. Regulatory authorities require that different production lots of any formulated drug product will have predictable properties, and that the product will have stability for a prolonged period to make its bioavailability and impurity profile constant throughout the expected duration of storage and use.

Amorphous drug substances can have properties that are advantages in the preparation of solid dosage forms, such as improved solubility, bioavailability, functional mechanics, or adhesivity. However, the increased reactivity of an amorphous solid, with a consequent high propensity to spontaneous transform to the crystalline state under certain conditions, such as relative humidity, applied force and temperature, may negatively affect the physical and chemical stability of their pharmaceutical preparations. Thus, the use of drugs and excipients in amorphous form represents both potential advantages and disadvantages to the formulator. Attempts have therefore been made to overcome the disadvantages by modulating the solid-state reactivity of amorphous substances, in terms of increasing or decreasing their reactivity. The various approaches used for the formulation of an amorphous material involve the use of dry granulation techniques for tableting, complexation, dry mixing, melt extrusion, co-precipitation, spray drying, co-milling, and others.

Retaining the drug in an amorphous form in the final dosage form generally improves the dissolution characteristics of the dosage form. A poster by S. E. Bartsch et al., “Melt Granulation of Solid Dispersions—Granulation Mechanism, Tableting and Dissolution Behavior,” presented at the 17^{th} Congress of the Austrian Pharmaceutical Society, Apr. 24-26, 2003, Graz, Austria, indicates that dissolution rates increase in the following order: pure drug substance<physical mixture<solid dispersion<melt granules<amorphous drug<tableted melt granules. This study compared tablets, made from glibenclamide granules produced by fluid-bed melt granulation, that used different granule sizes and were formed by different compression forces (between 10 and 20 kN), and found higher dissolution rates when the granules were larger, and when the compression forces were higher.

The various methods of producing an amorphous form of compounds include: spray drying; freeze drying (lyophilization); melt precipitation; vapor condensation; crash cooling from supercritical fluids, e.g. using Solution Enhanced Dispersion by Supercritical fluids (SEDS), Rapid Expansion of Supercritical Solution (RESS) processes, etc.; co-precipitation with suitable excipients such as sugars, acids, polymers, insoluble or enteric polymers, or surfactants to form solid dispersions; and molecular dispersions, co-precipitates or co-evaporates by melting or fusion, or from solvents including supercritical solvents.

It is known that amorphous materials frequently exhibit improved compression characteristics over the corresponding crystalline forms. For example, commercial grades of lactose are produced by a spray drying technique to introduce some amorphous content, which improves the compression force and hardness profiles of this tablet excipient. (A. H. Kibbe, Ed., Handbook of Pharmaceutical Excipients, 3rd Edition, American Pharmaceutical Association, Washington, D.C. USA, p. 276, 2000)

Amorphous materials do not exhibit the three-dimensional long-range order that is found in crystalline materials, but are structurally more similar to liquids where the arrangement of molecules is random. Amorphous solids are not crystalline and therefore do not give a definitive X-ray diffraction pattern. In addition they do not give rise to a melting point and tend to liquefy at some point beyond the glass transition point (B. C. Hancock and G. Zografi, “Characteristics and Significance of the Amorphous State in Pharmaceutical Systems,” Journal of Pharmaceutical Science, Vol. 86, pp. 1-12, 1997).

While the amorphous form of the drug has distinct physicochemical properties, it frequently has a persistent stability problem, in terms of maintaining its amorphous form during storage. Often the crystalline form of the drug has a lower free energy and thus, over time, the amorphous drug will tend to crystallize. The rate of crystallization may be influenced by storage conditions, such as temperature and humidity, as well as by the other constituents of the composition.

In the manufacture of drug substances, or in the processing of pharmaceutical solids, degrees of disorder through the formation of defects and amorphous regions are often observed. The amorphous state is mostly detected after lyophilization, spray drying, or milling. It results in a higher energy state than that for the crystalline state. This can provide more advantageous properties such as enhanced dissolution rate or better tableting properties. Often it is associated with increased chemical instability and difficulties in mixing and milling. Solid-state transformation upon storage is the most common and undesirable property since the driving force is kinetic, which is often difficult to suppress. Furthermore, depending on the conditions, metastable or stable forms may result. Amorphous forms are more hygroscopic and absorbed water plays the role of plasticizer, causing a lowering of the glass transition temperatures, resulting in an accelerated process of crystallization. The form of the eventual crystal is highly unpredictable. The change of the form of the drug substance affects the quality of the drug product, in terms of inconsistencies in the purity, identity and bioavailability of the drug product. Attempts have been made in the past to overcome these problems.

Published U.S. Patent Application 2003/0104063 A1 (Babcock et al.) teaches a pharmaceutical composition comprising a dispersion of a low-solubility drug and a matrix, combined with a concentration-enhancing polymer. At least a major portion of the drug is amorphous in the dispersion. The compositions improve the stability of the drug in the dispersion, and/or the bioavailability of the drug.

Published U.S. Patent Application 2003/0129250 A1 (Batycky et al.) reports an improved dissolution of poorly soluble drugs without sacrificing targeted flowability, wetability, selective agglomeration, annealing, yield or polymorphic stability. This is achieved by forming particles for oral drug delivery by spray drying a dilute solution of the poorly soluble drug and excipients. The particles comprise regions of poorly soluble drug wherein the dissolution rate enhancement is between about 2-fold and about 25-fold higher than that of the drug in bulk form.

It is always desirable to obtain a composition comprising a drug that is physically and chemically stable under typical storage conditions, can be formed via practical processing conditions, and that has a predictable bioavailability. These needs, and others that will become apparent to one of ordinary skill in the art, are met by the present invention, which is described in detail below.

### SUMMARY OF THE INVENTION

The invention comprises tablets comprising a drug substance that is susceptible to polymorphic conversion, the tablets having been formed by compression with forces sufficiently low to maintain the drug in its original polymorphic form

In one aspect, the invention includes a method of preparing a pharmaceutical dosage form, comprising: (a) forming a mixture comprising a drug that is susceptible to polymorphic change with pharmaceutically acceptable excipients; and (b) applying a pressure between about 0.2 and 5 tons to form minitablets.

In another aspect, the invention includes a method of preparing a pharmaceutical dosage form, comprising: (a) forming a mixture comprising a drug that is susceptible to polymorphic change with one or more pharmaceutically acceptable excipients; (b) applying a pressure between about 0.2 and 5 tons to form minitablets; and (c) optionally applying a coating to the minitablets.

In other aspects, the pressure applied to form minitablets is between about 0.2 and 3 tons, or about 2.5 tons.

### BRIEF DESCRIPTION OF THE DRAWINGS

FIG. 1 is a graphic depiction of the results of the experiment in Example 2.

FIG. 2 is an X-ray powder diffraction pattern for crystalline esomeprazole magnesium trihydrate.

FIG. 3 is an X-ray powder diffraction pattern for amorphous esomeprazole magnesium.

FIG. 4 is an X-ray powder diffraction pattern of tablets prepared according to Example 1.

FIG. 5 is an X-ray powder diffraction pattern of tablets containing amorphous esomeprazole magnesium, obtained following storage.

### DETAILED DESCRIPTION

This invention is useful for formulating dosage forms including any drug substance that is susceptible to polymorphic form conversion during formulation processing or during storage. Drug substances that have been otherwise stabilized against polymorphic changes, such as by combinations of the drug substances with crystallization inhibitors, or drugs that are distributed in a very fine particle form over the surfaces of excipient particles, can also benefit from use in the present invention.

The invention can be applied to crystalline forms of a drug, which are susceptible to polymorphic conversion from one crystalline form to a more thermodynamically stable different crystalline form, either spontaneously or while undergoing a customary formulation processing. Some of these drugs are vanlaxafine, valdecoxib, nateglinide, olanzapine, ezetimibe, and donepezil, but many others have thermodynamically unstable crystalline forms. Both free drug compounds and their salts, esters, etc. can be used in the invention.

The invention can be applied to any amorphous drug that is susceptible to conversion into a more thermodynamically stable crystalline form during processing or during storage. A few examples of such drugs are rosiglitazone, tegaserod, zolpidem, omeprazole, esomeprazole magnesium trihydrate, atorvastatin calcium, cetirizine dihydrochloride, fexofenadine hydrochloride, ziprasidone hydrochloride, donepezil hydrochloride, venlafexine hydrochloride, azithromycin, quinalapril hydrochloride, and clopidogrel. This list is not exhaustive, and the invention is not limited to use with any particular amorphous drug substance. In many instances, drug compounds can be present as the free compound, or as some salt or ester thereof, or in another form, and any of these that can be made amorphous will be suitable for use in the invention.

While any polymorphic form can be used in the invention, the following discussion will primarily describe the use of an amorphous form for preparing dosage forms. However, it should be understood that the overall concept and procedure will not differ substantially if the selected drug substance is a crystalline polymorph.

The term “substantially amorphous” in the context of this invention means that the substance has no greater than about 10 percent by weight crystalline content. The content which is crystalline can easily be determined by X-ray powder diffraction techniques, as is known in the art.

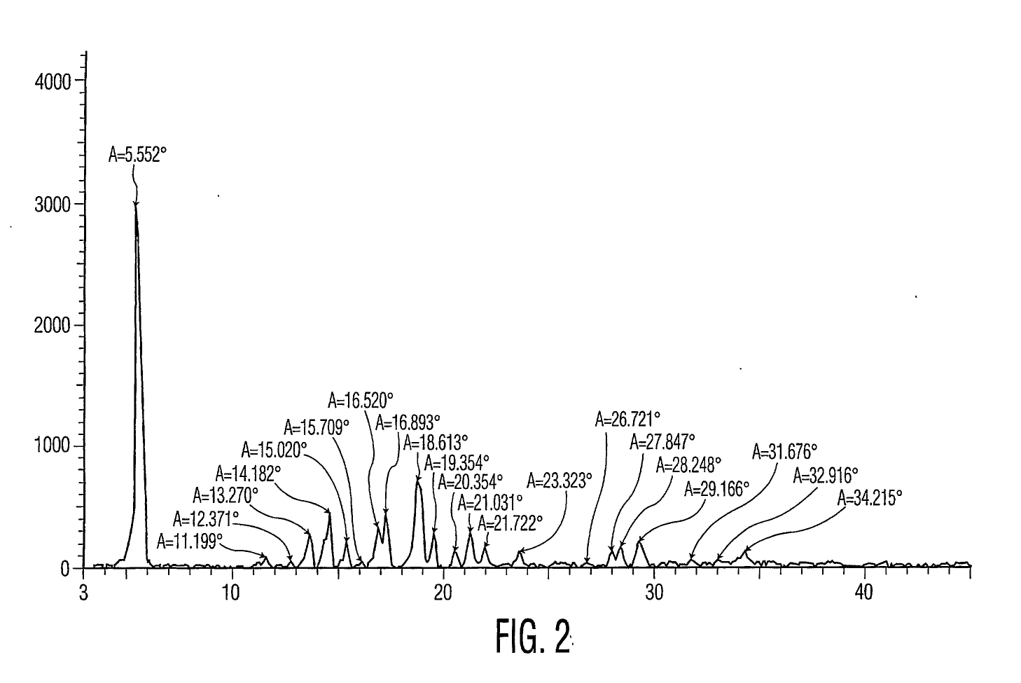

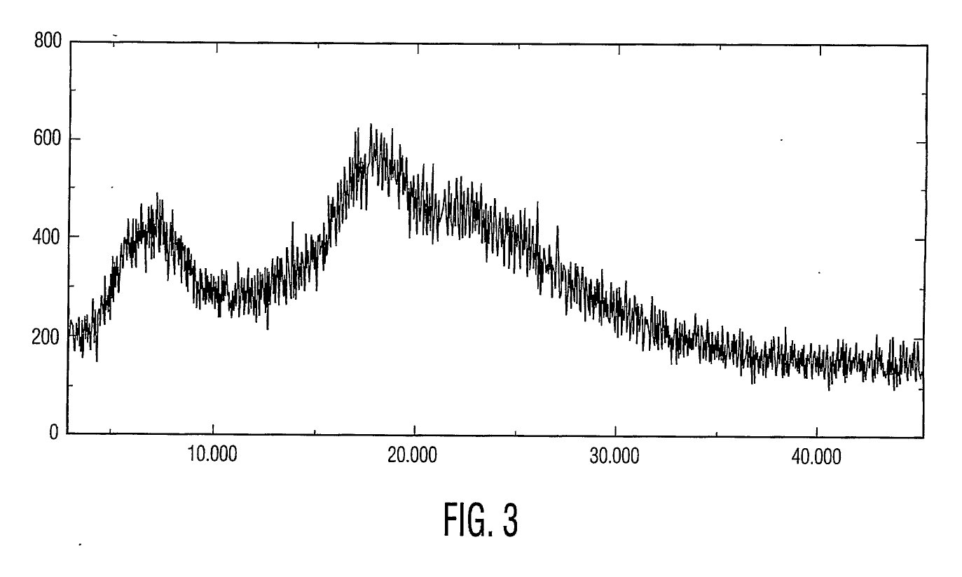

FIG. 2 is the X-ray powder diffraction pattern, using Cu K_{α1} radiation, of crystalline esomeprazole magnesium trihydrate. FIG. 3 is the X-ray powder diffraction pattern, using Cu K_{α1} radiation, of amorphous esomeprazole magnesium trihydrate. In each instance, the x-axis is 2θ, in degrees, and the y-axis is intensity. Using standard mixtures are prepared using known crystalline and amorphous materials, X-ray diffraction peak heights for varying crystalline contents can be calculated. Using this analytical technique, it is possible to detect about 2 percent by weight of crystalline content in an amorphous material sample.

The art is replete with reports of undesired conversion of the amorphous form of a substance to a crystalline form during conventional tableting procedures, as well as conversion in the presence of moisture. The present inventors in their attempt to formulate several amorphous drug substances have discovered that an amorphous drug substance, upon undergoing wet granulation, drug layering through coating, or compression in conventional tablet compression equipment, has shown changes in form from amorphous to crystalline. The presence of crystalline drug in a final dosage form can adversely affect its dissolution, when compared to a dosage form containing the pure amorphous form of the drug.

Surprisingly, the inventors have found that minitablets produced by the use of a reduced compression pressure do not show a significant conversion from the amorphous form of the drug.

To form the tablets of the invention, the drug substance is blended with one or more pharmaceutically acceptable carriers, fillers or extenders, such as starch, lactose, sucrose, glucose, mannitol, or silicic acid; binders, such as carboxymethylcellulose, alginates, gelatin, copolyvidonum (such as the PLASDONE™ S-630 copolymer of N-vinyl-2-pyrrolidone and vinyl acetate, sold by International Specialty Products, Wayne, N.J. U.S.A,), copolymers of ethylene oxide and propylene oxide such as Poloxamer 407, sucrose, or acacia; humectants, such as glycerol; disintegrants, such as starch, polyvinyl pyrrolidones, celluloses, formaldehyde-casein compounds, defatted soybean extracts, alginic acid, agar-agar, calcium carbonate, calcium phosphate, potato or tapioca starch or sodium carbonate; lubricants such as talc, calcium stearate, magnesium stearate or solid polyethylene glycol; solution retarding agents, such as paraffin; absorption accelerators, such as quaternary ammonium compounds; wetting agents, such as cetyl alcohol and glycerol monostearate; surfactants, such as sodium lauryl sulfate or docusate sodium; absorbents, such as kaolin or bentonite clay; and stabilizing agents. The pharmaceutical active may also be blended with buffering agents such as alkali metal carbonates and alkaline earth metal oxides. This listing is not exhaustive, many other functional components that are known in the art will also be useful in the present invention.

The mixture is then compressed, using any of the methods, such as applying pressure to powder contained in a die and punch assembly, that are well known in the art.

In some embodiments, the drug substance requires protection against acidic environments and formed tablets will be provided with an enteric coating. An “enteric coating” is a coating that is substantially insoluble at the acidic pH conditions of the stomach but is substantially soluble or water-permeable at the higher pH conditions of the intestines. In this invention, the enteric coating protects the tablet against contact with the acidic stomach environment but permits contact of the tablet with the more alkaline intestinal fluid. The enteric coating can be chosen to provide targeted release to a particular section of the intestine. For instance, an enteric coating can provide delivery to the duodenum (pH>5.5), to the jejunum (pH 6-7), or to the ileum (pH up to 7.5). Intermediate delivery points can be achieved by combining different coating materials or varying the thickness of the coating. Enteric coating materials include cellulose-based coatings, such as cellulose acetate phthalate and hydroxypropylmethyl cellulose phthalate, methacrylate-based coatings, polyvinyl acetate phthalate-based coatings, and shellac-based coatings. Many pre-formulated enteric coatings are commercially available, and any of these can be used in the present invention.

Further, some drug substances should be isolated from contact with enteric coating components that are themselves acidic in nature. In these situations, an intermediate coating, or subcoating, can be placed onto the formed tablets, before the enteric coating is applied. Useful ingredients for use in forming a subcoating include prolamines such as zein, crospovidone; croscarmellose sodium; cellulose derivatives such as hydroxypropyl methylcellulose, hydroxyethylcellulose, hydroxypropyl cellulose, or methylcellulose; gums such as seaweed extracts, plant extracts, plant exudates, plant seed extracts, and microbial fermentation products; starches including pregelatinized and modified starches; and synthetics such as carboxyvinyl polymers, including carbopols. Additional specific examples include alginates, pectins, low methoxy pectins, agar, carrageenan, plus arabic, tragacanth, karaya, ghatti, locust bean (carob), guar, dextran, xanthan, carrageenan, tara, Khaya grandfolia, gellan, Konjac mannan, galactomannan, funoran, acetan, welan, rhamsan, furcelleran, succinoglycan, scieroglycan, schizophylan, curdlan, pullulan, karaya and tamarind gums.

In some instances, particularly when it is desired to imprint identifying or other information on the coated or uncoated tablet, or where resistance to abrasion or humidity is required, a film outer coating will be applied. These film coatings commonly comprise polymers such as cellulose acetate phthalate, a cellulose ether, and the like, together in a fluid matrix with property-modifying ingredients such as plasticizers, surfactants, colorants and opacifiers, and others. Many formulated commercial products are available for applying a film coating.

In general, coatings may be applied by any techniques known in the art, such as pan coating (including perforated closed system pan coating), coacervation, or fluidized bed coating. The fluidized bed may contain a rotor insert and/or a Wurster column insert. The coatings can be generally classified according to their polymer base, such as: cellulose-based, including cellulose acetate phthalate, hydroxypropylmethyl cellulose phthalate, hydroxypropylmethyl cellulose, hydroxypropyl cellulose, hydroxypropylethyl cellulose, ethyl cellulose, methyl cellulose, microcrystalline cellulose; carrageenan; methacrylate- or methacrylic acid-based, such as methacrylic acid, methacrylate, acrylate, methacrylate, ethacrylate, methylmethacrylate, or copolymers thereof; or polyvinyl acetate phthalate-based. Typically, the polymer is combined with a solvent, such as water and/or an alcohol, and a plasticizer, such as polyethylene glycol, lactic acid, lactic acid, acetamide, glycerin, glyceryl monostearate, triacetin, sorbitol, triethyl citrate, polyvinylpyrrolidone, triethylene glycol, tricresyl phosphate, dibutyl tartrate, ethylene glycol monooleate, palmitic acid, stearic acid, oleic acid, or dibutyl sebacate. Optionally, one can also add any of the following elements: an anti-tack agent, an anti-foam agent, a filler, a surfactant, a colorant, a flavoring agent, and combinations of any two or more thereof.

Minitablets are inexpensive to produce and also save time and provide flexibility in designing dosage forms. Minitablets, which are for purposes of this application tablets of any shape having a maximum dimension no greater than about than 3 mm, are an interesting alternative in producing multiple-unit dosage forms. They can be made by ordinary tableting machines by direct compression, and have several advantages because of their production process and their product properties.

Minitablets show a resistance against densification and can be compressed to graded relative densities at reduced pressures. This increased densification under pressure leads to both higher permanent densification and higher elastic densification, which in turn results in higher elastic recovery. The inventors discovered that by using a compression pressure of only about 0.2 to 5 tons, preferably about 0.2 to 3 tons, such as about 2.5 tons, to produce tablets having diameters of about 1 mm to 3 mm, they were able to avoid the conversion of the amorphous drug substances into crystalline forms. It was also observed that the minitablets also have a reduced capping tendency when compared to conventional tablets.

The unit “ton” as used herein means kilonewtons (kN). This is consistent with usage of the term by manufacturers of tablet compression machinery, and is a unit of force that is applied to the material being compressed, such as with a punch and die.

The minitablets have moreover been shown to have several additional advantages over the conventional tablets. One advantage of minitablets lies in their precision of size, resulting in a high degree of dosage precision. Each individual minitablet meets all the requirements of a single-dose drug form, such as uniformity of mass and content. They can therefore be used individually, or a dosage form can be made by filling the tablets into capsules. Since capsules are usually made from gelatin and therefore have a permeability to water, in some cases a desiccant cartridge will be placed into containers of capsules so that the drug substance moisture exposure will be somewhat constant as capsules are removed periodically during use of the formulated product.

Representative useful formulations using the invention are as shown in the examples, which are presented to demonstrate certain aspects of the invention, but are not intended to limit the scope of the invention as it is defined in the appended claims.

### EXAMPLE 1

Dosage forms containing either 40 or 20 mg of esomeprazole were prepared as follows:

| | | |
|---|---|---|
| | mg for 40 | mg for 20 |
| Ingredients | mg Dose | mg Dose |

Tablet

| | | |
|---|---|---|
| Esomeprazole # | 40 | 20 |
| Mannitol | 242 | 264.2 |
| Low-substituted hydroxypropyl cellulose | 17.5 | 17.5 |
| Magnesium oxide | 20 | 20 |
| Sodium lauryl sulfate | 7 | 7 |
| Colloidal silicon dioxide | 3.5 | 3.5 |
| Sodium stearyl fumarate | 17.5 | 17.5 |
| Total | 352 | 352 |

Subcoating

| | | |
|---|---|---|
| Zein F 6000 | 12 | 12 |
| Eudragit ™ L 100-55 | 1.9 | 1.9 |
| Triethyl citrate | 0.19 | 0.19 |
| Cum. Total | 366.09 | 366.09 |

Enteric Coating

| | | |
|---|---|---|
| Eudragit ™ L 100-55 | 80.27 | 80.27 |
| Triethyl citrate | 8.06 | 8.06 |
| Glyceryl monostearate | 1.6 | 1.6 |
| Titanium dioxide | 1.6 | 1.6 |
| Cum. Total | 457.62 | 457.62 |

# Esomeprazole contained in the amorphous esomeprazole magnesium that was used for the formulation

Minitablets were produced by mixing amorphous esomeprazole magnesium with all of the other core ingredients, and directly compressing the dry mixture at 2.5 tons into cylindrical tablets having the diameter 2.5 mm, height 1.6-1.9 mm, and average weight 11 mg. The minitablets were then sub-coated with a solution of zein, triethyl citrate, and Eudragit™ L 100-55 (a copolymer of methacrylic acid and methyl methacrylate, sold by Röhm America LLC, Piscataway, N.J. USA) in isopropanol and water, and dried. Next, coated minitablets were enteric-coated using a solution of Eudragit™ L 100-55, glyceryl monostearate, and triethyl citrate in isopropanol, the solution containing suspended titanium dioxide, and dried. The enteric-coated minitablets were then filled into hard gelatin capsules. About 44 to 46 minitablets were contained in each capsule.

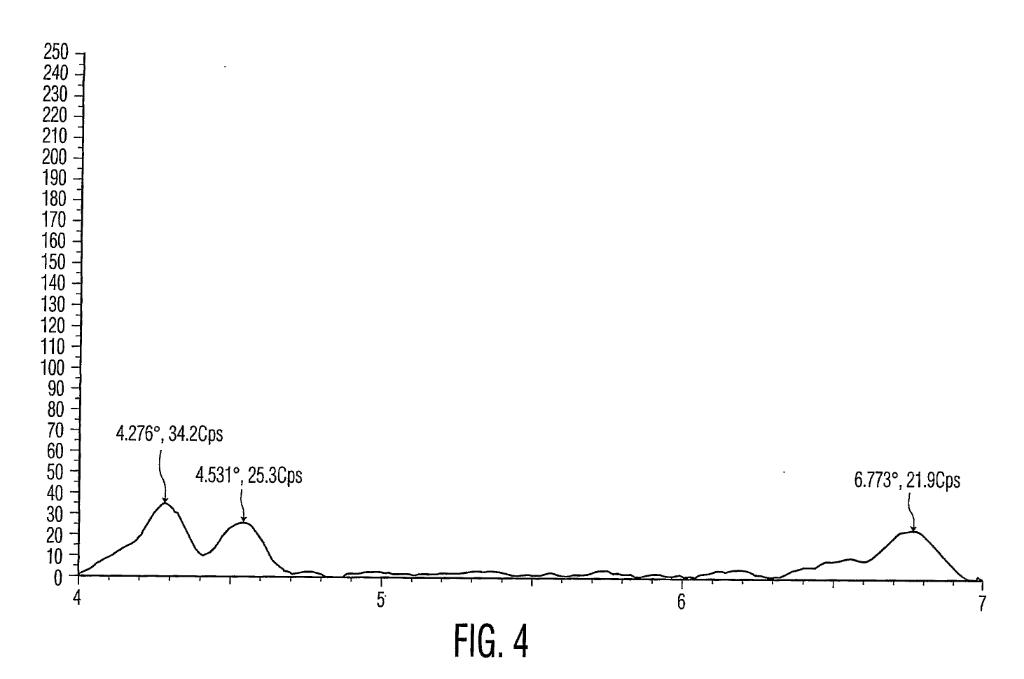

An uncoated compressed core was crushed and analyzed by X-ray powder diffraction, using Cu K_{α} radiation. The diffraction pattern is shown as FIG. 4.

### EXAMPLE 2

Tablets prepared according to the preceding example were tested to determine the dissolution characteristics, using the procedure in Method 711 of United States Pharmacopeia 24, The United States Pharmacopeial Convention, Inc., Rockville, Md. USA, 1999. As a comparison, tablets were similarly prepared using crystalline esomeprazole magnesium trihydrate.

The tablets were immersed in a pH 6.8 phosphate buffer solution at 37° C. and the solution stirred constantly during the test period. At intervals, samples of the solution were taken for analysis of the drug content. Release of the drug from the tablets into solution is shown in the following table:

Percent Drug Released

| Time, minutes | Crystalline | Amorphous |
|---|---|---|
| 0 | 0 | 0 |
| 10 | 15 | 14 |
| 20 | 35 | 54 |
| 30 | 53 | 86 |
| 45 | 70 | 91 |

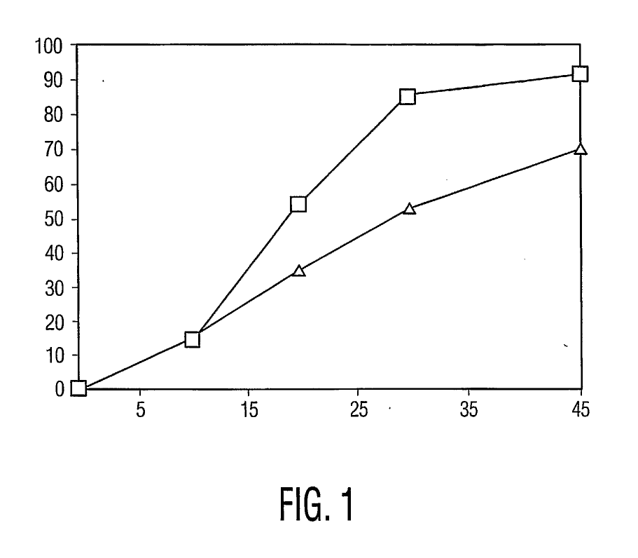

These results are plotted in the graph of FIG. 1, where the x-axis is time, in minutes, the y-axis is percent of the drug dissolved, data points for the amorphous drug tablets are represented by the squares, and data points for the crystalline drug are shown by triangles.

### EXAMPLE 3

Capsules containing 40 mg of esomeprazole were prepared using the following:

| | |
|---|---|
| Ingredient | mg/Capsule |

Tablet core

| | |
|---|---|
| Esomeprazole # | 40 |
| Mannitol | 245.1 |
| Low-substituted hydroxypropyl cellulose | 17.5 |
| Magnesium oxide | 20 |
| Colloidal silicon dioxide | 3.5 |
| Sodium lauryl sulfate | 7 |
| Sodium stearyl fumarate | 17.5 |

Subcoating Part 1

| | |
|---|---|
| Zein | 5.28 |

Subcoating Part 2

| | |
|---|---|
| Zein | 4.86 |
| Methacrylic acid copolymer, type C * | 2.08 |
| Triethyl citrate | 0.21 |

Enteric coating Part 1

| | |
|---|---|
| Methacrylic acid copolymer, type C * | 24.01 |
| Triethyl citrate | 6.01 |
| Glyceryl monostearate | 0.49 |
| Titanium dioxide | 0.49 |

Enteric coating Part 2

| | |
|---|---|
| Methacrylic acid copolymer, type C * | 33.16 |
| Sodium hydroxide | 0.44 |
| Triethyl citrate | 3.31 |
| Glyceryl monostearate | 0.66 |

# Esomeprazole equivalent contained in the amorphous esomeprazole magnesium that was used for the formulation
* EUDRAGIT ™ L 100-55 (copolymer of methacrylic acid and methyl methacrylate), sold by Röhm America LLC, Piscataway, New Jersey U.S.A.

Capsules were prepared by the following procedure:

Minitablets were prepared by mixing amorphous esomeprazole magnesium with all of the other tablet core ingredients and compressing at 2.5 tons into cylindrical shapes having diameters of 2.5 mm, heights of 1.6-1.9 mm, and weights averaging 11 mg.

The subcoating part 1 ingredient was dissolved in a mixture of water and isopropanol, then sprayed onto the minitablets in a rotating pan. Then the subcoating part 2 ingredients were prepared in a mixture of water and isopropanol, and applied to the zein-subcoated particles in a rotating pan. Solvents were removed by drying.

The subcoated particles were then coated with the enteric coating part 1 ingredients, in isopropanol, and the solvent was removed by drying. Finally, the particles were coated with the enteric coating part 2 ingredients, in water, and dried.

Enteric coated particles were filled into hard gelatin capsules, the capsules each containing an average of 44 to 46 particles.

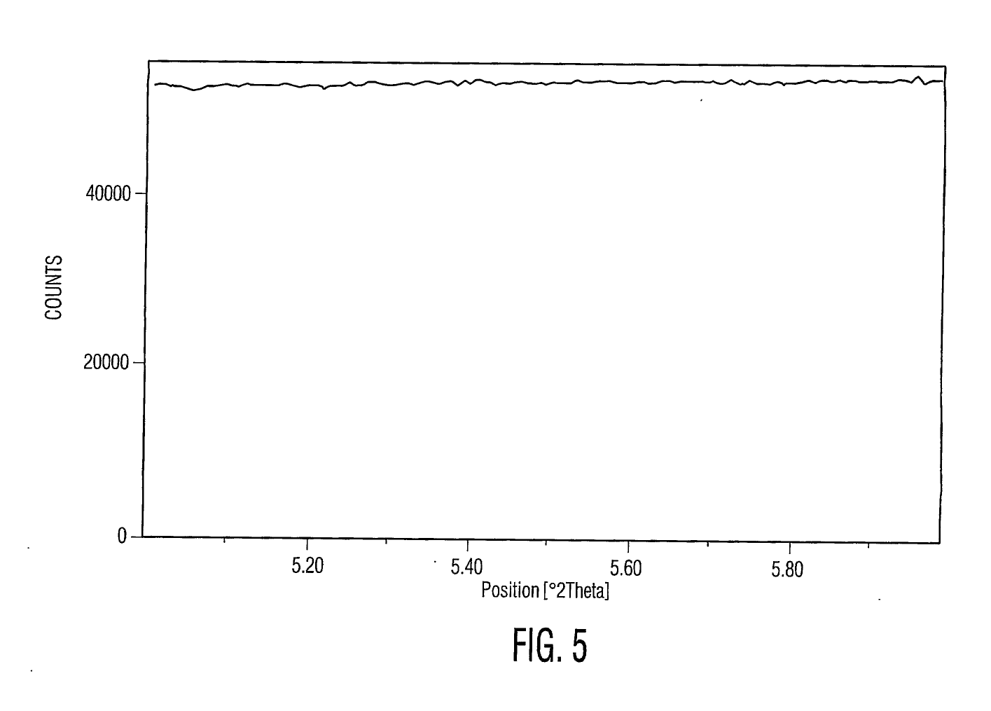

A representative sample of the capsules was placed into sealed high density polyethylene containers, or capsules were sealed into aluminum foil blisters, for storage at 40° C. and 75 percent relative humidity for two months. Following the storage period, the capsule contents were crushed and an X-ray powder diffraction pattern was obtained. A representative pattern over the interval 5-6° 2θ is shown as FIG. 5; this indicates the continued amorphous nature of the esomeprazole magnesium.

1. A tablet comprising a drug substance that is susceptible to polymorphic conversion, the tablet having been formed by compression with forces sufficiently low to maintain the drug in its original polymorphic form.

2. The tablet according to claim 1, wherein the drug substance is amorphous.

3. The tablet according to claim 2, wherein no greater than about 10 weight percent of the amorphous drug substance is crystalline.

4. The tablet according to claim 1, wherein compression is conducted between about 0.2 and about 5 tons.

5. The tablet according to claim 1, wherein compression is conducted between about 0.2 and about 3 tons.

6. The tablet according to claim 1, wherein a maximum tablet dimension is about 3 mm.

7. The tablet according to claim 1, wherein a maximum tablet dimension is about 1 mm to about 3 mm.

8. A pharmaceutical dosage form comprising a plurality of tablets prepared according to claim 1, contained within a capsule.

9. A pharmaceutical dosage form, comprising a plurality of particles formed by:
(a) mixing a drug substance that is susceptible to polymorphic conversion, with one or more pharmaceutically acceptable excipients;
(b) compressing the mixture at about 0.2 tons to about 5 tons, to form particles; and
(c) filling a plurality of the particles into a capsule.

10. The pharmaceutical dosage form according to claim 9, wherein the drug substance is amorphous.

11. The pharmaceutical dosage form according to claim 9, wherein no greater than about 10 weight percent of the drug substance is crystalline.

12. The pharmaceutical dosage form according to claim 9, wherein compressing is conducted at about 0.2 tons to about 3 tons.

13. The pharmaceutical dosage form according to claim 9, wherein a maximum particle dimension is about 3 mm.

14. A method of preparing a pharmaceutical dosage form, comprising:
(a) forming a mixture comprising a drug substance that is susceptible to polymorphic conversion, with one or more pharmaceutically acceptable excipients; and
(b) compressing the mixture at about 0.2 tons to about 5 tons, to form particles.

15. The method according to claim 14, wherein particles have a maximum dimension no greater than about 3 mm.

16. The method according to claim 14, wherein the drug is amorphous.

17. The method according to claim 14, further comprising applying a coating to the particles.

18. The method according to claim 14, wherein compression is conducted at about 0.2 tons to about 3 tons.

19. The method according to claim 14, wherein a maximum dimension is about 1 mm to about 3 mm.

20. The method according to claim 14, further comprising placing a plurality of particles into a capsule.

21. The tablet according to claim 1, wherein a drug substance comprises esomeprazole magnesium.

22. The pharmaceutical dosage form according to claim 9, wherein a drug substance comprises esomeprazole magnesium.

23. The method according to claim 14, wherein a drug substance comprises esomeprazole magnesium.
